SEQUENCE LISTING (71 sequences)

SEQ ID NO: 1 - DNA, 20 bp, Artificial Sequence
  Forward primer (Shigella)
aactggttacctgccgtgag

SEQ ID NO: 2 - DNA, 20 bp, Artificial Sequence
  Reverse primer (Shigella)
tggtgatggtggtggtaatg

SEQ ID NO: 3 - DNA, 23 bp, Artificial Sequence
  Forward primer (Clostridium)
aaaggragattaataccgcataa

SEQ ID NO: 4 - DNA, 20 bp, Artificial Sequence
  Reverse primer (Clostridium)
ttcttcctaatctctacgca

SEQ ID NO: 5 - DNA, 20 bp, Artificial Sequence
  Primer for AMPKa1
tgttccagcagatcctttcc

SEQ ID NO: 6 - DNA, 20 bp, Artificial Sequence
  Primer for AMPKa1
ataattgggtgagccacagc

SEQ ID NO: 7 - DNA, 21 bp, Artificial Sequence
  Primer for PPARa
tcttcacgatgctgtcctcct

SEQ ID NO: 8 - DNA, 20 bp, Artificial Sequence
  Primer for PPARa
ctatgtttagaaggccaggc

SEQ ID NO: 9 - DNA, 20 bp, Artificial Sequence
  Primer for GLUT2
attcgcctggatgagttacg

SEQ ID NO: 10 - DNA, 19 bp, Artificial Sequence
  Primer for GLUT2
cagcaaccatgaaccaagg

SEQ ID NO: 11 - DNA, 20 bp, Artificial Sequence
  Primer for G6Pase
gagtcttgtcaggcattgct

SEQ ID NO: 12 - DNA, 20 bp, Artificial Sequence
  Primer for G6Pase
gagtcttgtcaggcattgct

SEQ ID NO: 13 - DNA, 20 bp, Artificial Sequence
  Primer for Adiponectin
ttgcaagctctcctgttcct

SEQ ID NO: 14 - DNA, 20 bp, Artificial Sequence
  Primer for Adiponectin
tctccaggagtgccatctct

SEQ ID NO: 15 - DNA, 20 bp, Artificial Sequence
  Primer for Leptin
tgacaccaaaaccctcatca

SEQ ID NO: 16 - DNA, 19 bp, Artificial Sequence
  Primer for Leptin
agcccaggaatgaagtcca

SEQ ID NO: 17 - DNA, 20 bp, Artificial Sequence
  Primer for MCP-1
aggtccctgtcatgcttctg

SEQ ID NO: 18 - DNA, 20 bp, Artificial Sequence
  Primer for MCP-1
tctggacccattccttcttg

SEQ ID NO: 19 - DNA, 20 bp, Artificial Sequence
  Primer for TNFa
gccaccacgctcttctgcct

SEQ ID NO: 20 - DNA, 20 bp, Artificial Sequence
  Primer for TNFa
ggctgatggtgtgggtgagg

SEQ ID NO: 21 - DNA, 20 bp, Artificial Sequence
  Primer for IL-6
agttgccttcttgggactga

SEQ ID NO: 22 - DNA, 20 bp, Artificial Sequence
  Primer for IL-6
tccacgatttcccagagaac

SEQ ID NO: 23 - DNA, 20 bp, Artificial Sequence
  Primer for MUC2
acccgcactatgtcaccttc

SEQ ID NO: 24 - DNA, 20 bp, Artificial Sequence
  Primer for MUC2
gggatcgcagtggtagttgt

SEQ ID NO: 25 - DNA, 24 bp, Artificial Sequence
  Primer for GAPDH
gaaatcccatcaccatcttccagg

SEQ ID NO: 26 - DNA, 20 bp, Artificial Sequence
  Primer for GAPDH
gagccccagccttctccatg

SEQ ID NO: 27 - DNA, 26 bp, Artificial Sequence
  adaptor (27F Primer)
cctatcccctgtgtgccttggcagtc

SEQ ID NO: 28 - DNA, 4 bp, Artificial Sequence
  linker
tcag

SEQ ID NO: 29 - DNA, 20 bp, Artificial Sequence
  Forward 16s rRNA sequence (27F Primer)
agagtttgatcctggctcag

SEQ ID NO: 30 - DNA, 26 bp, Artificial Sequence
  adaptor
ccatctcatccctgcgtgtctccgac

SEQ ID NO: 31 - DNA, 10 bp, Artificial Sequence
  barcord (534R-1)
acgagtgcgt

SEQ ID NO: 32 - DNA, 17 bp, Artificial Sequence
  Reverse 16S rRNA sequence
attaccgcggctgctgg

SEQ ID NO: 33 - DNA, 10 bp, Artificial Sequence
  barcord (534R-2)
acgctcgaca

SEQ ID NO: 34 - DNA, 10 bp, Artificial Sequence
  barcord (534R-3)
agacgcactc

SEQ ID NO: 35 - DNA, 10 bp, Artificial Sequence
  barcord (534R-4)
agcactgtag

SEQ ID NO: 36 - DNA, 10 bp, Artificial Sequence
  barcord (534R-5)
atcagacacg

SEQ ID NO: 37 - DNA, 10 bp, Artificial Sequence
  barcord (534R-6)
atatcgcgag

SEQ ID NO: 38 - DNA, 10 bp, Artificial Sequence
  barcord (534R-7)
cgtgtctcta

SEQ ID NO: 39 - DNA, 10 bp, Artificial Sequence
  barcord (534R-8)
ctcgcgtgtc

SEQ ID NO: 40 - DNA, 10 bp, Artificial Sequence
  barcord (534R-9)
tagctctatc

SEQ ID NO: 41 - DNA, 10 bp, Artificial Sequence
  barcord (534R-10)
tctctatgcg

SEQ ID NO: 42 - DNA, 10 bp, Artificial Sequence
  barcord (534R-11)
tgatacgtct

SEQ ID NO: 43 - DNA, 10 bp, Artificial Sequence
  barcord (534R-12)
tatagacatc

SEQ ID NO: 44 - DNA, 10 bp, Artificial Sequence
  barcord (534R-13)
catagtagtg

SEQ ID NO: 45 - DNA, 10 bp, Artificial Sequence
  barcord (534R-14)
cgagagatac

SEQ ID NO: 46 - DNA, 10 bp, Artificial Sequence
  barcord (534R-15)
atacgacgta

SEQ ID NO: 47 - DNA, 10 bp, Artificial Sequence
  barcord (534R-16)
tcacgtacta

SEQ ID NO: 48 - DNA, 10 bp, Artificial Sequence
  barcord (534R-17)
cgtctagtac

SEQ ID NO: 49 - DNA, 10 bp, Artificial Sequence
  barcord (534R-18)
tctacgtagc

SEQ ID NO: 50 - DNA, 10 bp, Artificial Sequence
  barcord (534R-19)
tgtactactc

SEQ ID NO: 51 - DNA, 10 bp, Artificial Sequence
  barcord (534R-20)
acgactacag

SEQ ID NO: 52 - DNA, 10 bp, Artificial Sequence
  barcord (534R-21)
cgtagactag

SEQ ID NO: 53 - DNA, 10 bp, Artificial Sequence
  barcord (534R-22)
tacgagtatg

SEQ ID NO: 54 - DNA, 10 bp, Artificial Sequence
  barcord (534R-23)
tactctcgtg

SEQ ID NO: 55 - DNA, 10 bp, Artificial Sequence
  barcord (534R-24)
tagagacgag

SEQ ID NO: 56 - DNA, 10 bp, Artificial Sequence
  barcord (534R-25)
tcgtcgctcg

SEQ ID NO: 57 - DNA, 10 bp, Artificial Sequence
  barcord (534R-26)
acatacgcgt

SEQ ID NO: 58 - DNA, 10 bp, Artificial Sequence
  barcord (534R-27)
acgcgagtat

SEQ ID NO: 59 - DNA, 10 bp, Artificial Sequence
  barcord (534R-28)
actactatgt

SEQ ID NO: 60 - DNA, 10 bp, Artificial Sequence
  barcord (534R-29)
actgtacagt

SEQ ID NO: 61 - DNA, 10 bp, Artificial Sequence
  barcord (534R-30)
agactatact

SEQ ID NO: 62 - DNA, 10 bp, Artificial Sequence
  barcord (534R-31)
agcgtcgtct

SEQ ID NO: 63 - DNA, 10 bp, Artificial Sequence
  barcord (534R-32)
agtacgctat

SEQ ID NO: 64 - DNA, 10 bp, Artificial Sequence
  barcord (534R-33)
atagagtact

SEQ ID NO: 65 - DNA, 10 bp, Artificial Sequence
  barcord (534R-34)
cacgctacgt

SEQ ID NO: 66 - DNA, 10 bp, Artificial Sequence
  barcord (534R-35)
cagtagacgt

SEQ ID NO: 67 - DNA, 10 bp, Artificial Sequence
  barcord (534R-36)
cgacgtgact

SEQ ID NO: 68 - DNA, 10 bp, Artificial Sequence
  barcord (534R-37)
tacacacact

SEQ ID NO: 69 - DNA, 10 bp, Artificial Sequence
  barcord (534R-38)
tacacgtgat

SEQ ID NO: 70 - DNA, 10 bp, Artificial Sequence
  barcord (534R-39)
tacagatcgt

SEQ ID NO: 71 - DNA, 10 bp, Artificial Sequence
  barcord (534R-40)
tacgctgtct

(19) United States
(12) Patent Application Publication
(10) Pub. No.: US 2016/0160267 A1
(43) Pub. Date: Jun. 9, 2016

(54) COMPOSITION FOR EVALUATING OR PREDICTING PATIENTS THERAPEUTIC RESPONSE TO METHORMIN

(71) Applicant: SNU R&DB FOUNDATION, Seoul (KR)

(72) Inventors: Gwang Pyo KO, Seoul (KR); Hee Tae LEE, Uijeongbu (KR)

(21) Appl. No.: 14/905,327

(22) PCT Filed: Jul. 19, 2013
(86) PCT No.: PCT/KR2013/006506

§ 371 Date: Jan. 15, 2016

(51) Int. Cl. C12Q 1/68; A61K 31/155

(52) U.S. Cl. CPC C12Q 1/689; A61K 31/155; C12Q 2600/106

(57) ABSTRACT

The present invention relates to a biomarker for evaluating or predicting response of a patient to metformin, and use thereof. The present invention relates to a composition and a kit for evaluating or predicting therapeutic response of a patient to metformin, comprising an agent capable of detecting one or more microorganisms selected from the group consisting of Blautia, Shigella and Clostridium. And, the present invention relates to a method for detecting one or more microorganisms selected from the group consisting of Blautia, Shigella and Clostridium from the sample of a patient, so as to provide information required for evaluating or predicting response to metformin.

### TECHNICAL FIELD

The present invention relates to a biomarker for evaluating or predicting therapeutic response of a patient to metformin, and use thereof. More specifically, the present invention relates to a composition and a kit for evaluating or predicting therapeutic response of a patient to metformin, comprising an agent capable of detecting one or more microorganisms selected from the group consisting of Blautia, Shigella and Clostridium. The present invention also relates to a method for providing information required for evaluating or predicting therapeutic response of a patient to metformin.

### BACKGROUND ART

A biguanide compound metformin is a medicine that is most widely used for treating obesity, diabetes mellitus and metabolic syndrome up to date. Particularly, metformin is used a lot for a diabetic patient with obesity, particularly type 2 diabetic patient because of the weight loss effect, and it may prevent cardiovascular complications due to diabetes mellitus. The administration of metformin is known to control blood glucose by reducing glyconeogenesis in the liver, increasing insulin sensitivity, and increasing glucose uptake in the liver and muscle.

Obesity is occurred by energy balance collapse in human body due to genetic, environmental and psychological factors, and it has been already recognized as a “disease” to be cured worldwide, and the number of patients is continuously increasing due to change in life habit, industrialization and the like. According to the report of World Health Organization (WHO), worldwidely, about one billion populations have overweight, and 3 billion populations are obese patients with BMI of 30 kg/m² or more.

Diabetes mellitus is a disease with continued metabolic disorders including hyperglycemia due to lack of insulin action, and having high probability of the occurrence of vascular complications in the future, and it is divided into type 1 that is insulin-dependent and type 2 relating to both insulin resistance and insulin secretion dysfunction. Among them, type 2 diabetes mellitus currently occupies 80% or more of diabetic patients, the number of patients is continuously increasing due to population aging and change in life habit, and it is assumed that in industrialized countries, diabetic patients amount to 10% of the population.

Metabolic syndrome is characterized by high blood fat, hypertension, insulin resistance and central obesity (excessive adipose tissue in the abdominal region), and is a cause of all adult diseases, namely, obesity, diabetes mellitus, hypertension, and hyperlipidemia. Metabolic syndrome is a phenomenon that dangerous adult diseases such as artherosclerosis, hypertension, obesity, diabetes mellitus, hyperlipidemia and the like simultaneously occur in one person, and metabolic syndrome patients have a risk of sudden death due to cardiovascular disease, as well as chronic diseases such as diabetes mellitus and hypertension. Chronic metabolic disease due to increase in blood cholesterol and neutral fat is a fundamental cause of chronic metabolic diseases such as obesity, diabetes mellitus, metabolic syndrome and the like.

As the patients with obesity, diabetes mellitus and metabolic syndrome, important diseases in current society, are more and more increasing, use of metformin inevitably increases, and thus, evaluation and prediction of response or sensitivity of a patient to metformin is very important for establishing future treatment of a patient.

Meanwhile, drugs for treating disease exhibit medicinal effect in the body through various metabolisms, and since rumen microorganism communities closely affect the metabolism, intake and bioavailability of drug, even if the same drug is taken, the effect may be varied due to diversity in rumen microorganisms

Thus, response or sensitivity of a patient to drug for treating disease is varied according to an individual, and pharmaceutical effect may not be exhibited due to tolerance according to an individual. However, currently, there are no available methods for evaluating response of a patient to metformin at all.

### DISCLOSURE

### Technical Problem

Under these circumstances, the inventors examined change in rumen microorganism communities in mouse in which metabolic syndrome is induced through high-fat diet, confirmed that Blautia sp, Shigella sp and/or Clostridium sp may be used as a biomarker for evaluating and predicting therapeutic response of a patient to metformin, and completed the invention.

The present invention provides a biomarker for evaluating or predicting therapeutic response of a patient to metformin.

More specifically, the present invention provides a composition for evaluating or predicting therapeutic response of a patient to metformin, comprising an agent capable of detecting one or more microorganisms selected from the group consisting of Blautia, Shigella and Clostridium.

The present invention also provides a kit for evaluating or predicting therapeutic response of a patient to metformin, comprising the composition of the present invention.

The present invention also provides a method for providing information required for evaluating or predicting therapeutic response of a patient to metformin, comprising the steps of: detecting one or more microorganisms selected from the group consisting of Blautia, Shigella and Clostridium from the sample of a patient before and after administration of metformin; and determining that the patient has therapeutic response to metformin, if Blautia or Shigella increases or Clostridium decreases in the sample after the administration of metformin, compared to before the administration.

The present invention also provides a method for evaluating or predicting therapeutic response of a patient to metformin, comprising the steps of: detecting one or more microorganisms selected from the group consisting of Blautia, Shigella and Clostridium from the sample of a patient before and after administration of metformin; and determining that the patient has therapeutic response to metformin, if Blautia or Shigella increases or Clostridium decreases in the sample after the administration of metformin, compared to before the administration.

### Effect of the Invention

The present invention provides Blautia, Shigella and/or Clostridium as a novel biomarker for evaluating or predicting therapeutic response of a patient to metformin, and using the same, therapeutic response of a patient to metformin can be evaluated or predicted by simply gathering a sample.

### BRIEF DESCRIPTION OF DRAWINGS

FIG. 1 shows the experimental design for studying the effect of metformin treatment on rumen microorganism communities in mouse in which obesity, diabetes or metabolic syndrome is induced by high fat diet. In n=(x,y), x denotes the number of male mice, and y denotes the number of female mice.

FIG. 2a shows daily calorie intake of mouse.

FIG. 2b shows change in the body weight of male mouse during the experimental period, and FIG. 2c shows change in the body weight of female mouse during the experimental period. “▴” denotes a group treated with only high-fat diet without metformin administration for 28 weeks (HFD), “▪” denotes a group in which metformin is administered from 18 week while treated with high-fat diet for 28 weeks (HFD-M), “♦” denotes a group treated with high fat diet for 18 weeks and then treated with normal diet for the remaining 10 weeks (HI-D-ND), “_” denotes a group treated with only normal diet without metformin administration for 28 weeks (ND), and “•” denotes a group in which metformin is administered from 18 weeks while treated with normal diet for 28 weeks (ND-M).

FIG. 2d shows the results of measuring Glucose level at 12 week, which is before metformin administration, in mouse treated with high fat diet or normal diet, and FIG. 2e shows the results of measuring Glucose level at 21 week, which is after metformin administration, in mouse treated with high fat diet or normal diet.

FIG. 2f shows the results of oral glucose tolerance test (OGTT) 6 weeks after metformin administration, wherein Glucose tolerance is calculated and shown as AUC (area under the curve).

FIG. 2g shows the results of measuring glucose and insulin 3 weeks after metformin administration to calculate HOMA-IR (homeostatic model assessment-insulin resistance), FIG. 2h shows the results of measuring glucose and insulin 3 weeks after metformin administration to calculate HOMA-β (homeostatic model assessment-beta-cell).

FIG. 3a shows the results of measuring total cholesterol level at 16 week in high-fat diet-fed mouse and in normal diet-fed mouse.

FIG. 3b shows the results of measuring total cholesterol level 10 weeks after changing high-fat diet to normal diet, and 10 weeks after metformin administration while continuing high-fat diet.

FIG. 3c shows the results of measuring HDL (high-density lipoprotein) level at 16 week in high-fat diet-fed mouse and in normal diet-fed mouse.

FIG. 3d shows the results of measuring HDL level 10 weeks after changing high-fat diet to normal diet, and 10 weeks after metformin administration while continuing high-fat diet.

FIG. 4a shows the expression of metabolic and inflammatory biomarkers in the liver of male mouse.

FIG. 4b shows the expression of metabolic and inflammatory biomarkers in the liver of female mouse.

FIG. 4c shows the expression of metabolic and inflammatory biomarkers in the epididymal adipose tissue of male mouse.

FIG. 4d shows the expression of metabolic and inflammatory biomarkers in the epididymal adipose tissue of female mouse.

FIG. 5a shows the expression of MUC2 in small intestine, and FIG. 5b shows the expression of MUC5 in small intestine.

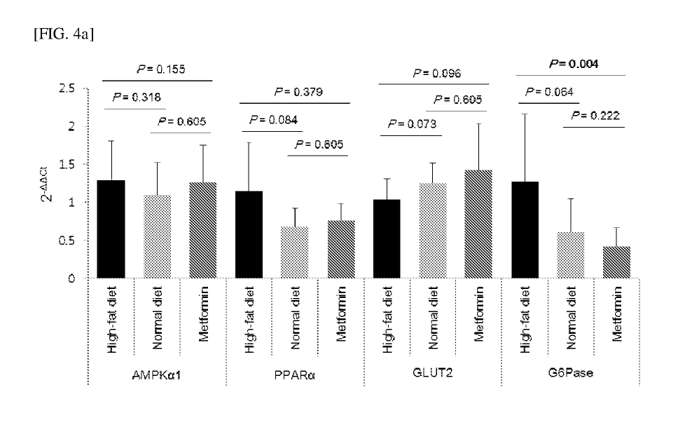

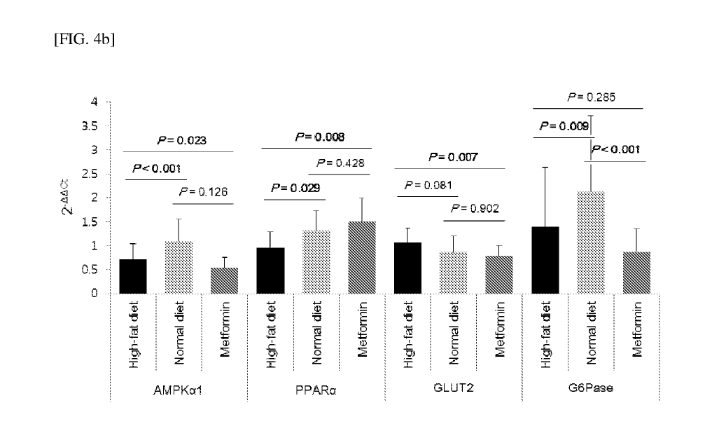

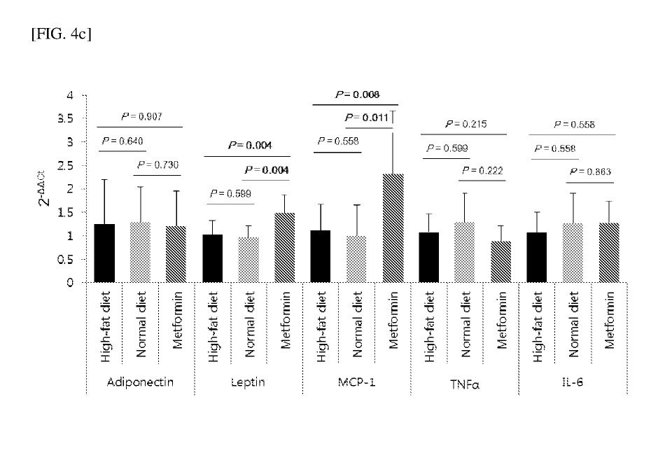

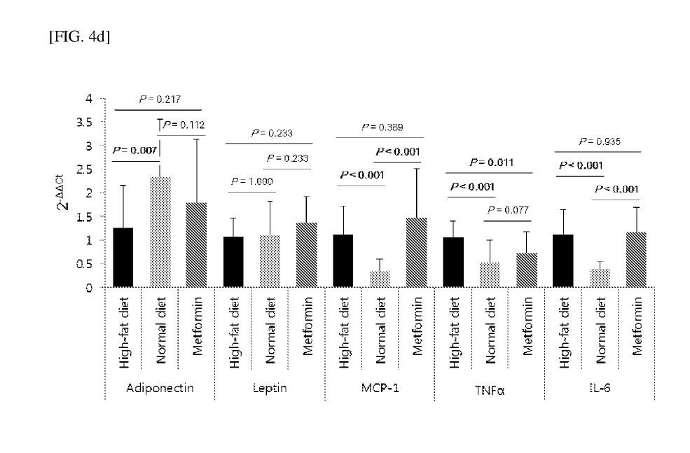

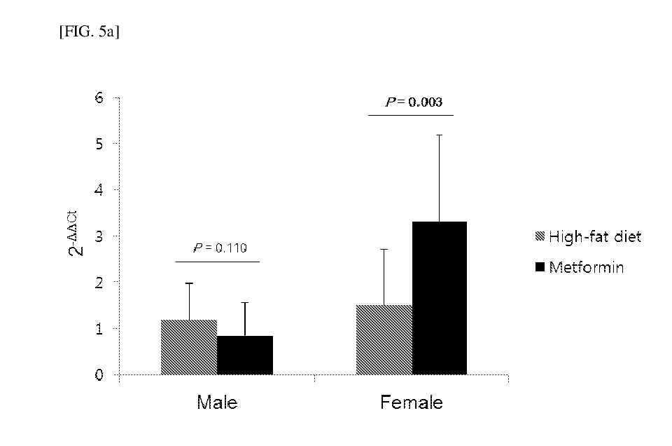

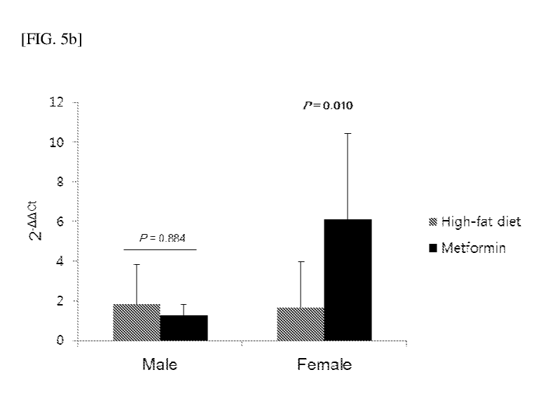

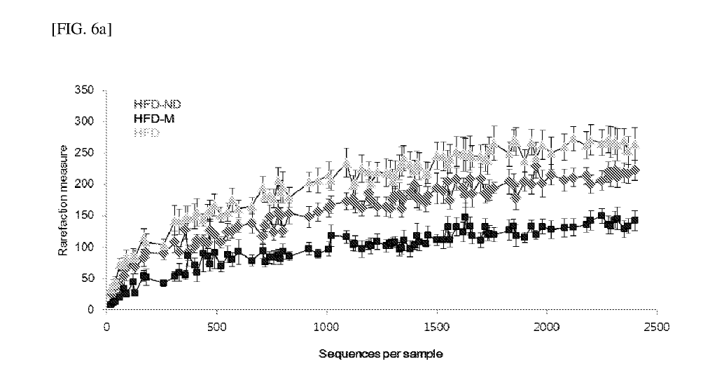

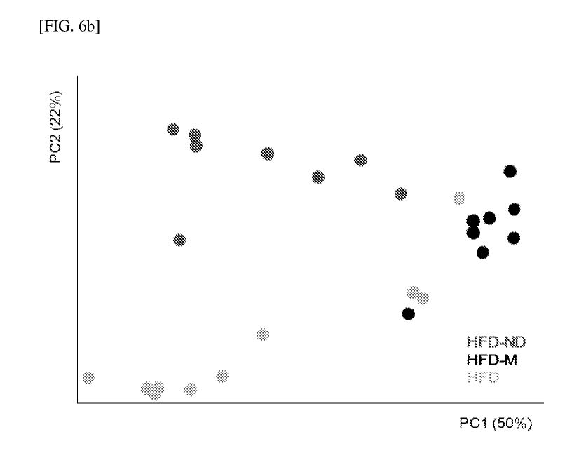

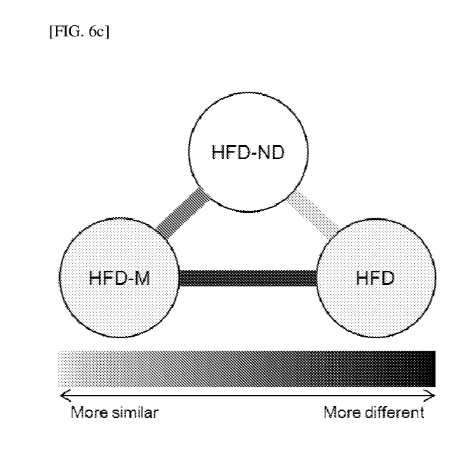

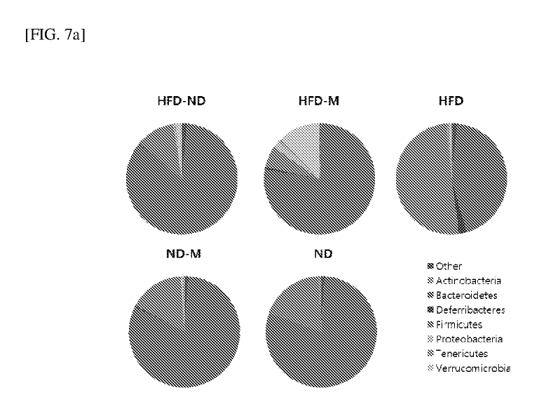

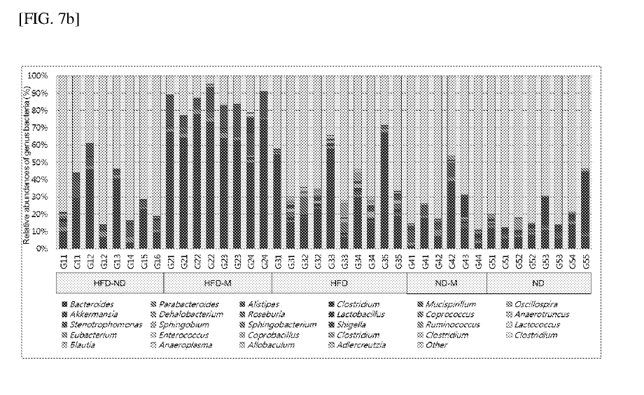

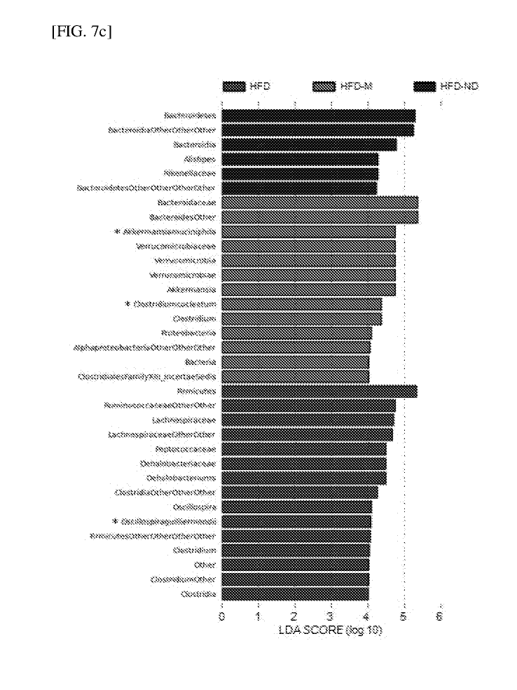

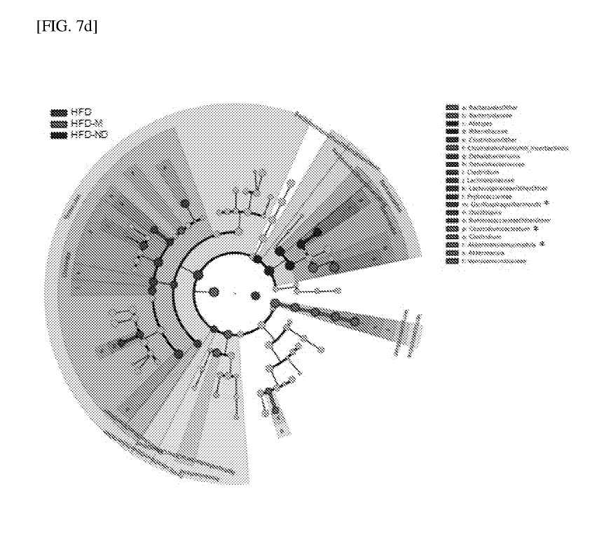

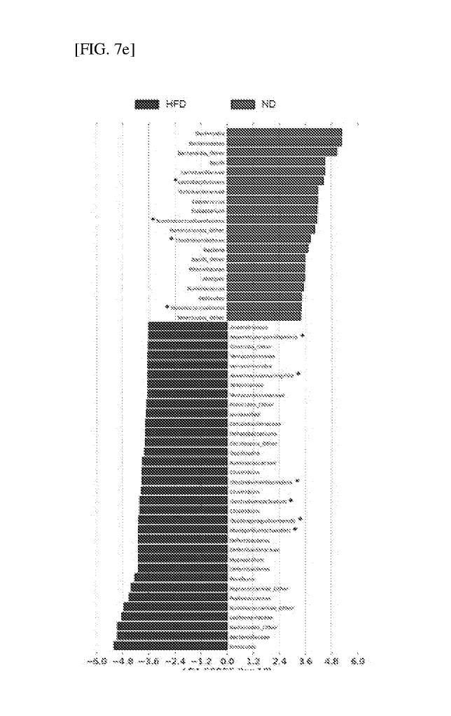

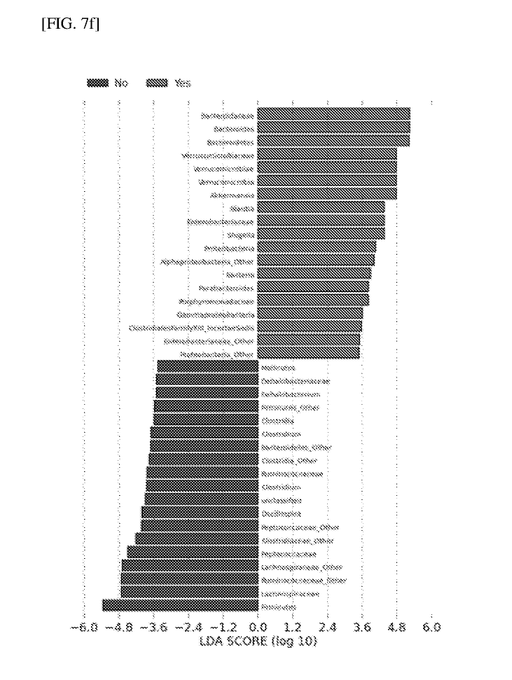

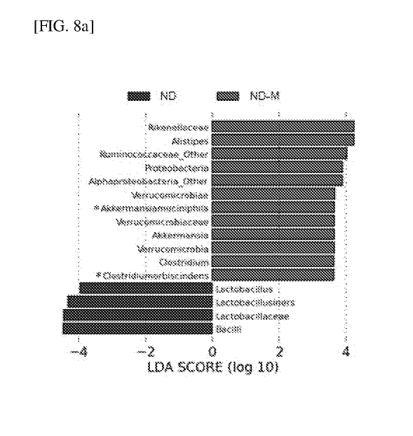

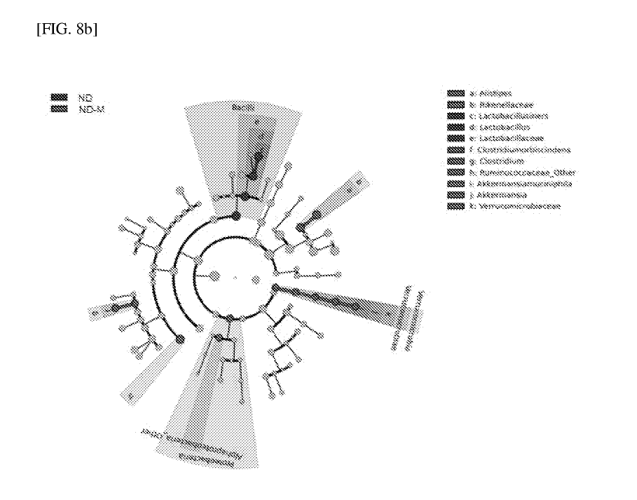

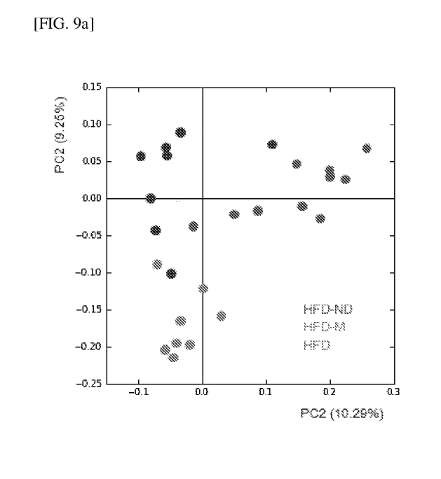

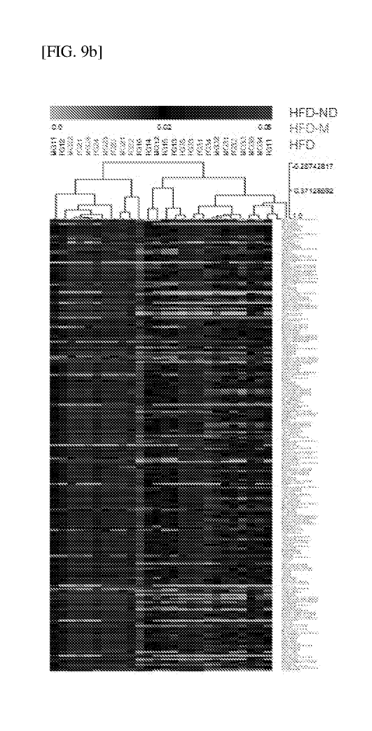

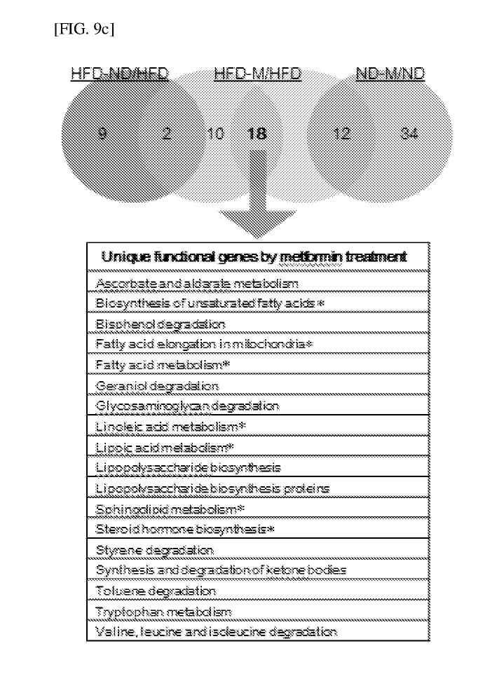

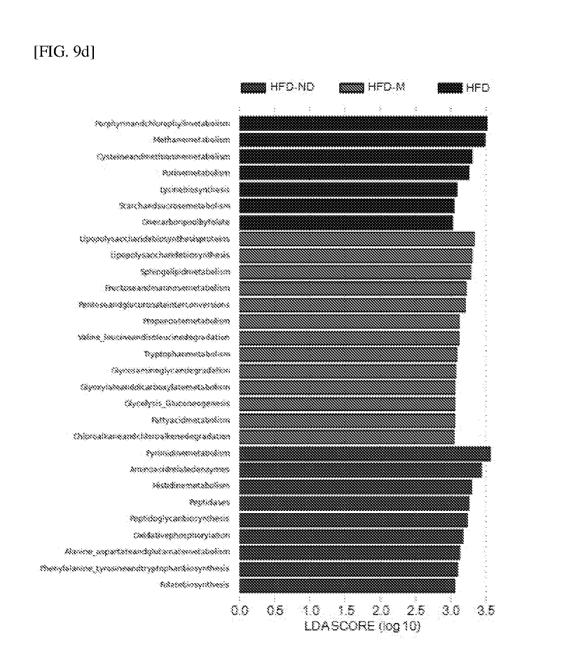

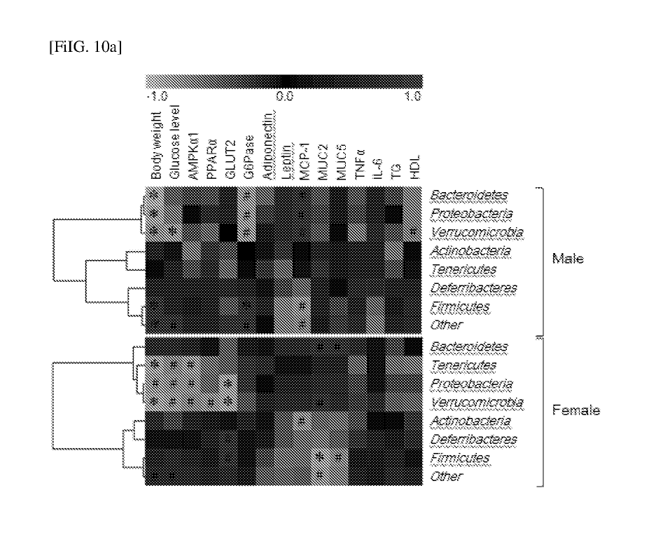

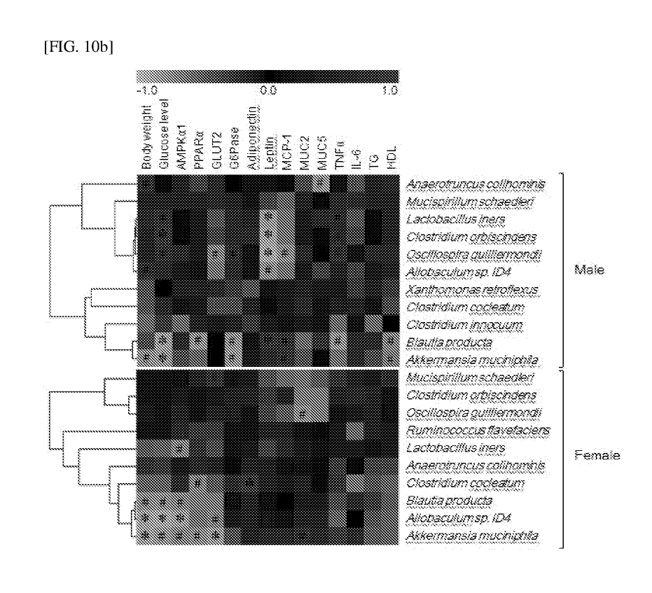

FIG. 4a to FIG. 5b comparatively analyze the amount of gene expression of internal control GAPDH and biomarkers using relative quantification method (2^{-ΔΔCt}(ΔΔCt=(C_{t.Target}−C_{t.GAPDH})_{Group1}−(C_{t.Target}−C_{t.GAPDH})_{Group2})). Statistical significance is assessed by Mann-Whitney U Test. For quantification, SYBR qPCR was repeatedly conduced three times.

FIG. 6a shows the rarefaction curve of microbial diversity from 40 feces samples of mice.

FIG. 6b shows bacterial communities using PCoA (principle coordinate analysis).

FIG. 6c visualizes a UniFrac distance between groups.

In FIGS. 6a to 6c, “HFD-ND” denotes a group in which high-fat diet is changed to normal diet, “HI-D-M” denotes a group to which metformin is administered while continuing high-fat diet, and “HFD” denotes a group to which only high-fat diet is treated without diet change.

FIG. 7a shows the results of classification based on 16 sRNA genes of microorganisms at phylum level.

FIG. 7b shows the results of classification based on 16 sRNA genes of microorganisms at genus level.

FIG. 7c shows LEfSe (LDA Effect Size) results of Kruskal-Wallis test between classes and Wilcoxon test between subclasses at P value of 0.05. “*” denotes the species of microorganism. Threshold on the logarithmic LDA score is 3.0.

FIG. 7d shows cladogram of Kruskal-Wallis test between classes and Wilcoxon test between subclasses at P value of 0.05. “*” denotes the species of microorganism.

FIG. 7e shows bacterial abundance between normal diet group and high-fat diet group without diet change as LEfSe. Threshold on the logarithmic LDA score is 3.0.

FIG. 7f shows bacterial abundance of the group to which metformin is administered while continuing high-fat diet, as LEfSe (LDA Effect Size) results of Kruskal-Wallis test between classes and Wilcoxon test between subclasses at P value of 0.05. Threshold on the logarithmic LDA score is 3.0.

In FIGS. 7a to 7f, “HFD-ND” denotes a group in which high-fat diet is changed to normal diet, “HI-D-M” denotes a group to which metformin is administered while continuing high-fat diet, “HFD” denotes a group to which only high-fat diet is treated without diet change, “ND-M” denotes a group to which metformin is administered while continuing normal diet, and “ND” denotes a group to which only normal diet is treated without diet change.

Total 7 phyla and 28 genera were identified in 40 feces samples gathered from male and female mice.

FIG. 8a shows the classification of microorganisms after metformin administration while continuing normal diet, as LDA score of Kruskal-Wallis test between classes and Wilcoxon test between subclasses at P value of 0.05. Threshold on the logarithmic LDA score is 3.0.

FIG. 8b shows the classification of microorganisms after metformin administration while continuing normal diet, as cladogram of Kruskal-Wallis test between classes and Wilcoxon test between subclasses at P value of 0.05.

In FIGS. 8a and 8b “*” denotes the species of microorganism, “ND” denotes a group to which metformin is administered while continuing normal diet, and “ND-M” denotes a group to which only normal diet is treated without metformin administration.

In FIGS. 9a to 9d, “HFD-ND” denotes a group in which high-fat diet is changed to normal diet, “HFD-M” denotes a group to which metformin is administered while continuing high-fat diet, “HFD” denotes a group to which only high-fat diet is treated without metformin administration, “ND-M” denotes a group to which metformin is administered while continuing normal diet, and “ND” denotes a group to which only normal diet is treated without metformin administration.

FIG. 9a shows KEGG pathways between different mouse groups using PCoA.

FIG. 9b visualizes heatmap of the relative amount of 245 KEGG pathways. Feces samples were hierarchically clustered by Pearson correlation.

FIG. 9c shows unique functional genes by metformin treatment.

FIG. 9d shows the significant amount of KEGG pathways by metformin administration as LEfSe. Among the total 245 KEGG pathways, 130 KEGG pathways were used to conduct LEfSe under metabolism. LEfSe is shown as the ranking of Kruskal-Wallis test between classes and Wilcoxon test between subclasses at P value of 0.05. Threshold on the logarithmic LDA score is 3.0.

FIGS. 10a and 10b show the correlation between microbial abundance and metabolism-related biomarkers as Spearman correlation. #: Significant in P<0.05, *: P<0.01.

### DETAILED DESCRIPTION OF THE EMBODIMENTS

In order to achieve the object, one embodiment of the invention relates to a composition for evaluating or predicting therapeutic response of a patient to metformin, comprising an agent capable of detecting one or more microorganisms selected from the group consisting of Blautia, Shigella and Clostridium.

Preferably, the agent capable of detecting microorganisms may be a microorganism-specific primer, probe, antisense oligonucleotide, aptamer or antibody.

More preferably, the primer may be a primer capable of amplifying 16S rRNA of microorganism.

Preferably, the patient may be a patient with obesity, diabetes, or metabolic syndrome.

Another embodiment of the invention relates to a kit for evaluating or predicting therapeutic response of a patient to metformin, comprising the above composition.

Another embodiment of the invention relates to a method for providing information required for evaluating or predicting therapeutic response of a patient to metformin, comprising the steps of: detecting one or more microorganisms selected from the group consisting of Blautia, Shigella and Clostridium from the sample of a patient before and after administration of metformin; and determining that the patient has therapeutic response to metformin, if Blautia or Shigella increases or Clostridium decreases in the sample after the administration of metformin, compared to before the administration.

Another embodiment of the invention relates to a method for evaluating or predicting therapeutic response of a patient to metformin, comprising the steps of: detecting one or more microorganisms selected from the group consisting of Blautia, Shigella and Clostridium from the sample of a patient before and after administration of metformin; and determining that the patient has therapeutic response to metformin, if Blautia or Shigella increases or Clostridium decreases in the sample after the administration of metformin, compared to before the administration.

Preferably, the step of detecting microorganisms comprises (a) extracting genome DNA from the sample of a patient to whom metformin is administered, (b) reacting the extracted genome DNA with a primer specific to one or more microorganisms selected from the group consisting of Blautia, Shigella and Clostridium to obtain reactant; and (c) amplifying the reactant.

More preferably, the step of amplifying the reactant may be conducted through polymerase chain reaction.

Preferably, the step (c) may further comprise comparing the amount of the amplified product with the amplified product of the sample before the administration of metformin.

Preferably, the sample of a patient may be a feces sample.

Preferably, the patient may be a patient with obesity, diabetes or metabolic syndrome.

Hereinafter, the present invention will be explained in detail.

The present invention is based on the discovery that when metformin is administered to mouse in which obesity, diabetes or metabolic syndrome is induced by high-fat diet, among rumen microorganism communities, Blautia and Shigella communities significantly increase, and Clostridium community specifically decreases, and is characterized by providing one or more microorganisms selected from the group consisting of Blautia, Shigella and Clostridium as a biomarker for evaluating or predicting response of a patient to metformin.

In the specific example of the invention, in order to evaluate or predict therapeutic response of a patient to metformin, metformin was administered to a mouse in which obesity, diabetes or metabolic syndrome was induced by high-fat diet to confirm change in rumen microorganism communities during the process of improving the symptoms of obesity, diabetes or metabolic syndrome. For this, microorganism communities were examined through pyrosequencing analysis of the variable region (V2-V3) of bacterial 16S rRNA gene.

As the result, after administering metformin to a mouse in which obesity, diabetes or metabolic syndrome is induced by high-fat diet, bacterial diversity generally decreased, and bacteria pertaining to phylum Proteobacteria and phylum Verrucomicrobia significantly increased. Particularly, it was confirmed that at genus level, Akkermansia, Shigella, and Blautia increased, while Clostridium significantly decreased.

Thus, the present invention can evaluate or predict therapeutic response of a patient to metformin by detecting Blautia, Shigella and/or Clostridium from the sample of a patient, and for this, provides a composition, a kit and a method for detecting Blautia, Shigella and/or Clostridium.

As used herein, the term “evaluating or predicting therapeutic response” means determining whether or not pathological condition will be maintained, improved or aggravated due to the effect of medicine after administering the medicine to a patient. If pathological condition is improved by the administration of medicine, treatment will be carried out by continuously administering the medicine; if pathological condition is maintained by the administration of medicine, prognosis will be observed while slowly administering the medicine; and if pathological condition is aggravated by the administration of medicine, the administration of the medicine will be discontinued.

According to one preferable embodiment, if Blautia and Shigella communities increase and Clostridium community decreases after administering metformin to a patient with obesity, diabetes or metabolic syndrome, metformin will be continuously administered to carry out treatment of obesity, diabetes or metabolic syndrome; and if there are no changes in Blautia, Shigella and Clostridium communities by the administration of metformin, prognosis will be observed while slowly administering metformin. And, if Blautia and Shigella communities decrease and Clostridium community increases after the administration of metformin, the administration of metformin will be discontinued. Furthermore, whether or not metformin will be administered to a patient with obesity, diabetes and metabolic syndrome, and dose thereof will be determined according to the degree of change in Blautia, Shigella and/or Clostridium.

As used herein, “Blautia” taxonomically means microorganisms consisting of species pertaining to genus Blautia, or a community thereof. And, microorganisms with preferably 70% or more, more preferably 80% or more, even more preferably 90% or more, most preferably 95% or more sequence homology to previously reported Blautia, when comparing 16s rRNA sequence, as well as previously reported strains are included in the scope of the present invention.

As used herein, “Shigella” taxonomically means microorganisms consisting of species pertaining to genus Shigella, or a community thereof. And, microorganisms with preferably 70% or more, more preferably 80% or more, even more preferably 90% or more, most preferably 95% or more sequence homology to previously reported Shigella, when comparing 16s rRNA sequence, as well as previously reported strains are included in the scope of the present invention.

As used herein, “Clostridium” taxonomically means microorganisms consisting of species pertaining to genus Clostridium, or a community thereof. And, microorganisms with preferably 70% or more, more preferably 80% or more, even more preferably 90% or more, most preferably 95% or more sequence homology to previously reported Clostridium, when comparing 16s rRNA sequence, as well as previously reported strains are included in the scope of the present invention.

As used herein, “obesity” means a state wherein adipose tissues were excessively accumulated due to an imbalance between calorie intake and consumption, and in a patient with obesity, adipose tissues are well-developed, and the number and the size of adipocytes are remarkably increased compared to normal person. In general, if body mass index (value obtained by dividing body weight (kg) by the square of height (m)) is 25 or more, it is diagnosed as obesity, but this is just one representative standard, and the scope of obesity of the present invention is not limited thereto.

As used herein, “diabetes mellitus” means a disease characterized by lack of insulin produced in beta cells of pancreas or insulin resistance and hyperglycemia occurred due to both defects. In general, diabetes mellitus is divided into insulin dependent diabetes mellitus (IDDM; Type I) and non-insulin dependent diabetes mellitus (NIDDM; Type II) occurred due to insulin resistance and impaired insulin secretion, and preferably, diabetes mellitus of the present invention may be non-insulin dependent diabetes mellitus. In general, “metabolic syndrome” means a syndrome showing risk factors such as hypertriglyceridemia, hypertension, abnormal glucose metabolism, abnormal blood coagulation and obesity together, but in the present invention, it is not limited thereto, and means a disease showing risk factors such as hyperlipidemia, hypertension, abnormal glucose metabolism, abnormal blood coagulation, cardiovascular atherosclerosis, and obesity.

As used herein, “an agent capable of detecting microorganisms” means substance that can be used to detect the existence of Blautia, Shigella and/or Clostridium, which is a biomarker of the present invention capable of evaluating or predicting therapeutic response of a patient to metformin in a sample. For example, it may be a primer, probe, antisense oligomucleotide, aptamer or antibody, and the like, capable of specifically detecting organic biomolecules such as protein, nucleic acid, lipid, glycolipid, glycoprotein or saccharides (monosaccharides, disaccharides, oligosaccharides, and the like), and the like, specifically existing in Blautia, Shigella and/or Clostridium.

Preferably, the agent capable of detecting microorganisms may be a primer capable of detecting Blautia, Shigella and/or Clostridium. It is preferable that the primer specifically detects the genome sequence of Blautia, Shigella and/or Clostridium, without specific binding to the genome sequences of other microorganisms. More specifically, it may be a primer capable of amplifying 16S rRNA of one or more microorganisms selected from the group consisting of Blautia, Shigella and Clostridium. For specific example, the primer pair specific to Shigella is represented by SEQ ID NO. 1 and SEQ ID NO. 2, and the primer pair specific to Clostridium is represented by SEQ ID NO. 3 and SEQ ID NO. 4 (Table 1)

TABLE 1

| | | Nucleotide sequence |
|---|---|---|
| SEQ ID NO | name | (5′-3′) |
| 1 | Forward primer | AACTGGTTACCTGCCGTGAG |
| 2 | Reverse primer | TGGTGATGGTGGTGGTAATG |
| 3 | Forward primer | AAAGGRAGATTAATACCGCATAA |
| 4 | Reverse primer | TTCTTCCTAATCTCTACGCA |

“Metformin” is biguanide antidiabetics, and particularly, is the most important drug for treatment of diabetes mellitus type 2, and the chemical name is N,N-Dimethylimidodicarbonimidic diamide. It is also important drug for a patient with overweight and obesity. As used herein, “metformin” means all drugs that can be used for diabetes mellitus, obesity, metabolic syndrome-related diseases, including the above drug and derivatives thereof.

The term “primer” means 7 to 50 nucleic acid sequence that has a short free 3′ hydroxyl group, can form a base pair with a complementary template, and functions as a starting point for template strand replication. The primer is commonly synthesized, but can be used from naturally produced nucleic acid. The sequence of the primer is not necessarily exactly the same as the sequence of a template, and it has only to be sufficiently complementary so as to be hybridized to the template. The primer can initiate DNA synthesis in the presence of reagents for polymerization reaction (namely, DNA polymerase or reverse transcriptase) and different 4 kinds of nucleoside triphosphates, in an appropriate buffer solution at appropriate temperature. In the present invention, therapeutic response of a patient to metformin may be evaluated or predicted by PCR amplification using sense and antisense primers of Blautia, Shigella and/or Clostridium nucleotide sequences. The PCR conditions, and the lengths of sense and antisense primers may be modified based on the information known in the art. Preferably, the primer of the present invention may be a primer capable of amplifying 16s rRNA of Blautia, Shigella and/or Clostridium.

As used herein, the tem “16s rRNA” means rRNA constituting 30S subunit of ribosome of prokaryote, wherein most nucleotide sequence is highly conserved, while high nucleotide sequence diversity is shown in partial sections. Particularly, since there is little diversity between the same kinds, while diversity appears between different kinds, prokaryotes may be usefully identified by comparing the sequence of 16S rRNA.

According to one preferable embodiment, the primer may be used to amplify 16S rRNA sequence conserved in Blautia, Shigella and/or Clostridium, and the existence of Blautia, Shigella and/or Clostridium may be detected by confirming whether or not a desired product is produced as the results of sequence amplification. For sequence amplification using primer, various methods known in the art may be used. For example, polymerase chain reaction (PCR), reverse transcription-polymerase chain reaction (RT-PCR), multiplex PCR, touchdown PCR, hot start CPR, nested PCR, booster PCR, real-time PCR, differential display PCR (DD-PCR), rapid amplification of cDNA ends (RACE), inverse polymerase chain reaction, vectorette PCR, TAIL-PCR (thermal asymmetric interlaced PCR), ligase chain reaction, repair chain reaction, transcription-mediated amplification, replication of self-sustaining nucleotide sequence, selective amplification of target nucleotide sequence, and the like may be used, but not limited thereto.

The composition for evaluating or predicting therapeutic response of a patient to Metformin, comprising an agent capable of detecting one or more microorganisms selected from the group consisting of Blautia, Shigella and Clostridium may be provided in the form of a kit for evaluating or predicting therapeutic response of a patient to Metformin.

The kit of the present invention may comprise an agent such as a primer, probe, antisense oligonucleotide, aptamer, antibody, and the like, for detecting Blautia, Shigella and/or Clostridium, and one or more kind of other constitutional compositions, solutions or devices suitable for the analysis method.

For example, the kit comprising a primer specific to Blautia, Shigella and/or Clostridium may be a kit comprising essential elements for conducting an amplification reaction such as PCR and the like. For example, the kit for PCR may comprise a test tube or other appropriate container, a reaction buffer solution, enzyme such as deoxynucleotide (dNTPs), Taq-polymerase and reverse transcriptase, DNase, RNase inhibitor, DEPC-water, sterilized water, and the like.

And, the present invention provides a method for providing information required for evaluating or predicting therapeutic response of a patient to Metformin, comprising the steps of: (a) detecting one or more microorganisms selected from the group consisting of Blautia, Shigella and Clostridium from the sample of a patient before and after administration of Metformin; and (b) determining that the patient has therapeutic response to Metformin, if Blautia or Shigella increases or Clostridium decreases in the sample after the administration of Metformin, compared to before the administration.

And, the present invention provides a method for evaluating or predicting therapeutic response of a patient to Metformin, comprising the steps of: (a) detecting one or more microorganisms selected from the group consisting of Blautia, Shigella and Clostridium from the sample of a patient before and after administration of Metformin; and (b) determining that the patient has therapeutic response to Metformin, if Blautia or Shigella increases or Clostridium decreases in the sample after the administration of Metformin, compared to before the administration.

According to preferable example, the method may comprise the steps of extracting genome DNA from the sample of a patient to whom Metformin is administered, reacting the extracted genome DNA with a primer specific to one or more microorganisms selected from the group consisting of Blautia, Shigella and Clostridium to obtain reactant; and amplifying the reactant.

The “sample of a patient” is gathered from the body of a patient to whom metformin is administered, and includes samples such as tissue, cell, whole blood, serum, plasma, salvia or urine, and preferably, may be a feces sample of a patient. Wherein, “feces” means a sample including rumen microorganisms, which is the remainder of food not utilized in the body.

A method for extracting genome DNA from the sample of a patient may be conducted using common technology known in the art, and primer specific to Blautia, Shigella and/or Clostridium is as explained above.

In the step of “amplifying the reactant”, the method of amplifying the reactant may be conducted using common amplification technology known in the art, for example, polymerase chain reaction (PCR), reverse transcription-polymerase chain reaction (RT-PCR), multiplex PCR, touchdown PCR, hot start CPR, nested PCR, booster PCR, real-time PCR, differential display PCR (DD-PCR), rapid amplification of cDNA ends (RACE), inverse polymerase chain reaction, vectorette PCR, TAIL-PCR (thermal asymmetric interlaced PCR), ligase chain reaction, repair chain reaction, transcription-mediated amplification, replication of self-sustaining nucleotide sequence, selective amplification of target nucleotide sequence, and the like, but not limited thereto.

Hereinafter, the present invention will be explained in detail with reference to the following examples. However, these examples are only to illustrate the invention, and the scope of the invention is not limited thereto.

### Example 1

### Animal Model

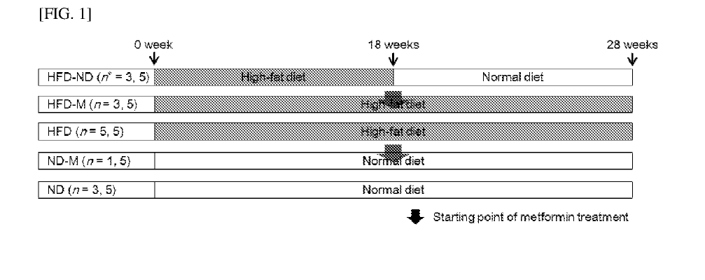

Male and female C57BL/6 species of the same age of the week were purchased from Orientbio. Diet with 60% of the total calorie being fat (TD.06416, Harlan Laboratories Inc.) was fed for 28 weeks to induce obesity and diabetes, and thereafter, metformin (D150959, Sigma-Aldrich) was orally administered at a dose of 300 mg/kg daily for 10 weeks. As control groups, a group to which normal diet with 5% of the total calorie being fat (Rodent NIH-31 Auto, Zeigler Bros., Inc.) was fed and a group in which high-fat diet was changed to normal diet were simultaneously tested to comparatively analyze the effect of metformin. All the experiments were deliberated by Institutional Animal Care and Use Committees and progressed. A basic model is as shown in FIG. 1.

### Example 2

### Metabolism-Related Index

Basically, body weight and fasting glucose were confirmed once a week. To confirm impaired glucose tolerance, oral glucose tolerance test (OGTT) was conducted. And, total cholesterol, high-density lipoprotein (HDL), low-density lipoprotein (LDL) were analyzed in serum, and insulin concentration in blood was measured using flow cytometry. To grasp the expression degree of metabolic and inflammatory factors, liver, epididymal adipose tissue, small intestine were homogenized to extract total RNA, which was quantitatively analyzed by polymerase chain reaction (PCR). In the liver, AMPKα1 (AMP-activated protein kinase alpha 1), PPARα (Peroxisome proliferator-activated eceptor alpha), GLUT2 (Glucose transporter 2), G6Pase (Glucose 6-phosphatase) were measured; in the adipose tissue, adiponectin, leptin, MCP-1 (Monocyte chemoattractant protein-1), TNFα (Tumor necrosis factor alpha) IL-6 (Interleukin-6) were measured; and in the small intestine, MUC2 and MUC5 (Mucin genes) were measured. For comparative analysis of the expression degree, relative quantification using housekeeping gene GAPDH was applied, and statistical significance was confirmed with Mann-Whitney U test. The primers used are summarized in Table 2. As the primer for MUC5AC, QuantiTect® Primer Assay (Cat. no.: QT01161104, Qiagen) was used.

TABLE 2

| | | Nucleotide sequence |
|---|---|---|
| SEQ ID NO | Target gene | (5′ - 3′) |
| 5 | AMPKα1 | TGTTCCAGCAGATCCTTTCC |
| 6 | AMPKα1 | ATAATTGGGTGAGCCACAGC |
| 7 | PPARα | TCTTCACGATGCTGTCCTCCT |
| 8 | PPARα | CTATGTTTAGAAGGCCAGGC |
| 9 | GLUT2 | ATTCGCCTGGATGAGTTACG |
| 10 | GLUT2 | CAGCAACCATGAACCAAGG |
| 11 | G6Pase | GAGTCTTGTCAGGCATTGCT |
| 12 | G6Pase | GAGTCTTGTCAGGCATTGCT |
| 13 | Adiponectin | TTGCAAGCTCTCCTGTTCCT |
| 14 | Adiponectin | TCTCCAGGAGTGCCATCTCT |
| 15 | Leptin | TGACACCAAAACCCTCATCA |
| 16 | Leptin | AGCCCAGGAATGAAGTCCA |
| 17 | MCP-1 | AGGTCCCTGTCATGCTTCTG |
| 18 | MCP-1 | TCTGGACCCATTCCTTCTTG |
| 19 | TNFα | GCCACCACGCTCTTCTGCCT |
| 20 | TNFα | GGCTGATGGTGTGGGTGAGG |
| 21 | IL-6 | AGTTGCCTTCTTGGGACTGA |
| 22 | IL-6 | TCCACGATTTCCCAGAGAAC |
| 23 | MUC2 | ACCCGCACTATGTCACCTTC |
| 24 | MUC2 | GGGATCGCAGTGGTAGTTGT |
| 25 | GAPDH | GAAATCCCATCACCATCTTCCAGG |
| 26 | GAPDH | GAGCCCCAGCCTTCTCCATG |

### Example 3

### Tissue

Epididymis adipose tissue, liver, small intestine, pancreas, and blood were extracted, and among them, liver, small intestine, and pancreas were fixed to 4% paraformaldehyde to conduct hematoxylin and eosin (H&E) staining. Briefly, tissue was cut to 4 μm, stained with alum haematoxylin and rinsed with water flow. Thereafter, the colour was changed with 0.3% acid alcohol, followed by additional staining with eosin, dehydrating and fixing. The degree of steatosis and inflammation of the tissue was read by pathologist.

### Example 4

### Analysis of Rumen Microbiome

Total genome DNA of bacteria in the feces sample was extracted using a kit. The V2 region and the V3 region of 16S rRNA gene were amplified through PCR using bar coded universal primer as described in Table 3 and Table 4, and pyrosequencing was conducted using GS-FLX system. For the secured nucleotide sequence, data was analyzed using QIIME (Quantitative Insights Into Microbial Ecology) 1.5.0 (http://qiime.sourceforge.net). Before the sequence analysis, noise removal of the sequence data set was conducted, and low quality sequence less than 200 bp was removed. To the nucleotide sequence, OUT (Operational taxonomic units) was assigned based on 97% analogous level to conduct system analysis. For the nucleotide sequence obtained from each feces sample, UniFrac, PCoA (Principal Coordinate Analysis), LEfSe (LDA Effect Size) analysis were conducted for comparative analysis. And, based on the KEGG (Kyoto Encyclopedia of Genes and Genomes) pathway data from microbiome, PICRUSt (Phylogenetic Investigation of Communities by Reconstruction of Unobserved States) was conducted to predict a specifically increasing metabolic pathway.

TABLE 3

| | | SEQ | |
|---|---|---|---|
| | classifi- | ID | Nucleotide sequence |
| name | cation | NO | (5′ - 3′) |
| 27F | Adapter | 27 | CCTATCCCCTGTGTGCCTTGGCAGTC |
| Primer | Linker | 28 | TCAG |
| | Forward 16s | 29 | AGAGTTTGATCCTGGCTCAG |
| | rRNA | | |

TABLE 4

| Name (ID) | classification | SEQ ID NO | Nucleotide sequence (5′ - 3′) |
|---|---|---|---|
| 534R-1 | Adapter | 30 | CCATCTCATCCCTGCGTGTCTCCGAC |
| (MID-1) | Linker | 28 | TCAG |
| | Barcode | 31 | ACGAGTGCGT |
| | Backward 16s rRNA | 32 | ATTACCGCGGCTGCTGG |
| 534R-2 | Adapter | 30 | CCATCTCATCCCTGCGTGTCTCCGAC |
| (MID-2) | Linker | 28 | TCAG |
| | Barcode | 33 | ACGCTCGACA |
| | Backward 16s rRNA | 32 | ATTACCGCGGCTGCTGG |
| 534R-3 | Adapter | 30 | CCATCTCATCCCTGCGTGTCTCCGAC |
| (MID-3) | Linker | 28 | TCAG |
| | Barcode | 34 | AGACGCACTC |
| | Backward 16s rRNA | 32 | ATTACCGCGGCTGCTGG |
| 534R-4 | Adapter | 30 | CCATCTCATCCCTGCGTGTCTCCGAC |
| (MID-4) | Linker | 28 | TCAG |
| | Barcode | 35 | AGCACTGTAG |
| | Backward 16s rRNA | 32 | ATTACCGCGGCTGCTGG |
| 534R-5 | Adapter | 30 | CCATCTCATCCCTGCGTGTCTCCGAC |
| (MID-5) | Linker | 28 | TCAG |
| | Barcode | 36 | ATCAGACACG |
| | Backward 16s rRNA | 32 | ATTACCGCGGCTGCTGG |
| 534R-6 | Adapter | 30 | CCATCTCATCCCTGCGTGTCTCCGAC |
| (MID-6) | Linker | 28 | TCAG |
| | Barcode | 37 | ATATCGCGAG |
| | Backward 16s rRNA | 32 | ATTACCGCGGCTGCTGG |
| 534R-7 | Adapter | 30 | CCATCTCATCCCTGCGTGTCTCCGAC |
| (MID-7) | Linker | 28 | TCAG |
| | Barcode | 38 | CGTGTCTCTA |
| | Backward 16s rRNA | 32 | ATTACCGCGGCTGCTGG |
| 534R-8 | Adapter | 30 | CCATCTCATCCCTGCGTGTCTCCGAC |
| (MID-8) | Linker | 28 | TCAG |
| | Barcode | 39 | CTCGCGTGTC |
| | Backward 16s rRNA | 32 | ATTACCGCGGCTGCTGG |
| 534R-9 | Adapter | 30 | CCATCTCATCCCTGCGTGTCTCCGAC |
| (MID-102) | Linker | 28 | TCAG |
| | Barcode | 40 | TAGCTCTATC |
| | Backward 16s rRNA | 32 | ATTACCGCGGCTGCTGG |
| 534R-10 | Adapter | 30 | CCATCTCATCCCTGCGTGTCTCCGAC |
| (MID-10) | Linker | 28 | TCAG |
| | Barcode | 41 | TCTCTATGCG |
| | Backward 16s rRNA | 32 | ATTACCGCGGCTGCTGG |
| 534R-11 | Adapter | 30 | CCATCTCATCCCTGCGTGTCTCCGAC |
| (MID-11) | Linker | 28 | TCAG |
| | Barcode | 42 | TGATACGTCT |
| | Backward 16s rRNA | 32 | ATTACCGCGGCTGCTGG |
| 534R-12 | Adapter | 30 | CCATCTCATCCCTGCGTGTCTCCGAC |
| (MID-103) | Linker | 28 | TCAG |
| | Barcode | 43 | TATAGACATC |
| | Backward 16s rRNA | 32 | ATTACCGCGGCTGCTGG |
| 534R-13 | Adapter | 30 | CCATCTCATCCCTGCGTGTCTCCGAC |
| (MID-13) | Linker | 28 | TCAG |
| | Barcode | 44 | CATAGTAGTG |
| | Backward 16s rRNA | 32 | ATTACCGCGGCTGCTGG |
| 534R-14 | Adapter | 30 | CCATCTCATCCCTGCGTGTCTCCGAC |
| (MID-14) | Linker | 28 | TCAG |
| | Barcode | 45 | CGAGAGATAC |
| | Backward 16s rRNA | 32 | ATTACCGCGGCTGCTGG |
| 534R-15 | Adapter | 30 | CCATCTCATCCCTGCGTGTCTCCGAC |
| (MID-15) | Linker | 28 | TCAG |
| | Barcode | 46 | ATACGACGTA |
| | Backward 16s rRNA | 32 | ATTACCGCGGCTGCTGG |
| 534R-16 | Adapter | 30 | CCATCTCATCCCTGCGTGTCTCCGAC |
| (MID-16) | Linker | 28 | TCAG |
| | Barcode | 47 | TCACGTACTA |
| | Backward 16s rRNA | 32 | ATTACCGCGGCTGCTGG |
| 534R-17 | Adapter | 30 | CCATCTCATCCCTGCGTGTCTCCGAC |
| (MID-17) | Linker | 28 | TCAG |
| | Barcode | 48 | CGTCTAGTAC |
| | Backward 16s rRNA | 32 | ATTACCGCGGCTGCTGG |
| 534R-18 | Adapter | 30 | CCATCTCATCCCTGCGTGTCTCCGAC |
| (MID-18) | Linker | 28 | TCAG |
| | Barcode | 49 | TCTACGTAGC |
| | Backward 16s rRNA | 32 | ATTACCGCGGCTGCTGG |
| 534R-19 | Adapter | 30 | CCATCTCATCCCTGCGTGTCTCCGAC |
| (MID-19) | Linker | 28 | TCAG |
| | Barcode | 50 | TGTACTACTC |
| | Backward 16s rRNA | 32 | ATTACCGCGGCTGCTGG |
| 534R-20 | Adapter | 30 | CCATCTCATCCCTGCGTGTCTCCGAC |
| (MID-20) | Linker | 28 | TCAG |
| | Barcode | 51 | ACGACTACAG |
| | Backward 16s rRNA | 32 | ATTACCGCGGCTGCTGG |
| 534R-21 | Adapter | 30 | CCATCTCATCCCTGCGTGTCTCCGAC |
| (MID-21) | Linker | 28 | TCAG |
| | Barcode | 52 | CGTAGACTAG |
| | Backward 16s rRNA | 32 | ATTACCGCGGCTGCTGG |
| 534R-22 | Adapter | 30 | CCATCTCATCCCTGCGTGTCTCCGAC |
| (MID-22) | Linker | 28 | TCAG |
| | Barcode | 53 | TACGAGTATG |
| | Backward 16s rRNA | 32 | ATTACCGCGGCTGCTGG |
| 534R-23 | Adapter | 30 | CCATCTCATCCCTGCGTGTCTCCGAC |
| (MID-23) | Linker | 28 | TCAG |
| | Barcode | 54 | TACTCTCGTG |
| | Backward 16s rRNA | 32 | ATTACCGCGGCTGCTGG |
| 534R-24 | Adapter | 30 | CCATCTCATCCCTGCGTGTCTCCGAC |
| (MID-24) | Linker | 28 | TCAG |
| | Barcode | 55 | TAGAGACGAG |
| | Backward 16s rRNA | 32 | ATTACCGCGGCTGCTGG |
| 534R-25 | Adapter | 30 | CCATCTCATCCCTGCGTGTCTCCGAC |
| (MID-25) | Linker | 28 | TCAG |
| | Barcode | 56 | TCGTCGCTCG |
| | Backward 16s rRNA | 32 | ATTACCGCGGCTGCTGG |
| 534R-26 | Adapter | 30 | CCATCTCATCCCTGCGTGTCTCCGAC |
| (MID-26) | Linker | 28 | TCAG |
| | Barcode | 57 | ACATACGCGT |
| | Backward 16s rRNA | 32 | ATTACCGCGGCTGCTGG |
| 534R-27 | Adapter | 30 | CCATCTCATCCCTGCGTGTCTCCGAC |
| (MID-27) | Linker | 28 | TCAG |
| | Barcode | 58 | ACGCGAGTAT |
| | Backward 16s rRNA | 32 | ATTACCGCGGCTGCTGG |
| 534R-28 | Adapter | 30 | CCATCTCATCCCTGCGTGTCTCCGAC |
| (MID-28) | Linker | 28 | TCAG |
| | Barcode | 59 | ACTACTATGT |
| | Backward 16s rRNA | 32 | ATTACCGCGGCTGCTGG |
| 534R-29 | Adapter | 30 | CCATCTCATCCCTGCGTGTCTCCGAC |
| (MID-29) | Linker | 28 | TCAG |
| | Barcode | 60 | ACTGTACAGT |
| | Backward 16s rRNA | 32 | ATTACCGCGGCTGCTGG |
| 534R-30 | Adapter | 30 | CCATCTCATCCCTGCGTGTCTCCGAC |
| (MID-30) | Linker | 28 | TCAG |
| | Barcode | 61 | AGACTATACT |
| | Backward 16s rRNA | 32 | ATTACCGCGGCTGCTGG |
| 534R-31 | Adapter | 30 | CCATCTCATCCCTGCGTGTCTCCGAC |
| (MID-31) | Linker | 28 | TCAG |
| | Barcode | 62 | AGCGTCGTCT |
| | Backward 16s rRNA | 32 | ATTACCGCGGCTGCTGG |
| 534R-32 | Adapter | 30 | CCATCTCATCCCTGCGTGTCTCCGAC |
| (MID-32) | Linker | 28 | TCAG |
| | Barcode | 63 | AGTACGCTAT |
| | Backward 16s rRNA | 32 | ATTACCGCGGCTGCTGG |
| 534R-33 | Adapter | 30 | CCATCTCATCCCTGCGTGTCTCCGAC |
| (MID-33) | Linker | 28 | TCAG |
| | Barcode | 64 | ATAGAGTACT |
| | Backward 16s rRNA | 32 | ATTACCGCGGCTGCTGG |
| 534R-34 | Adapter | 30 | CCATCTCATCCCTGCGTGTCTCCGAC |
| (MID-34) | Linker | 28 | TCAG |
| | Barcode | 65 | CACGCTACGT |
| | Backward 16s rRNA | 32 | ATTACCGCGGCTGCTGG |
| 534R-35 | Adapter | 30 | CCATCTCATCCCTGCGTGTCTCCGAC |
| (MID-35) | Linker | 28 | TCAG |
| | Barcode | 66 | CAGTAGACGT |
| | Backward 16s rRNA | 32 | ATTACCGCGGCTGCTGG |
| 534R-36 | Adapter | 30 | CCATCTCATCCCTGCGTGTCTCCGAC |
| (MID-36) | Linker | 28 | TCAG |
| | Barcode | 67 | CGACGTGACT |
| | Backward 16s rRNA | 32 | ATTACCGCGGCTGCTGG |
| 534R-37 | Adapter | 30 | CCATCTCATCCCTGCGTGTCTCCGAC |
| (MID-37) | Linker | 28 | TCAG |
| | Barcode | 68 | TACACACACT |
| | Backward 16s rRNA | 32 | ATTACCGCGGCTGCTGG |
| 534R-38 | Adapter | 30 | CCATCTCATCCCTGCGTGTCTCCGAC |
| (MID-38) | Linker | 28 | TCAG |
| | Barcode | 69 | TACACGTGAT |
| | Backward 16s rRNA | 32 | ATTACCGCGGCTGCTGG |
| 534R-39 | Adapter | 30 | CCATCTCATCCCTGCGTGTCTCCGAC |
| (MID-39) | Linker | 28 | TCAG |
| | Barcode | 70 | TACAGATCGT |
| | Backward 16s rRNA | 32 | ATTACCGCGGCTGCTGG |
| 534R-40 | Adapter | 30 | CCATCTCATCCCTGCGTGTCTCCGAC |
| (MID-40) | Linker | 28 | TCAG |
| | Barcode | 71 | TACGCTGTCT |
| | Backward 16s rRNA | 32 | ATTACCGCGGCTGCTGG |

### Experimental Results

### 1. Change in Metabolism-Related Index after Administration of Metformin

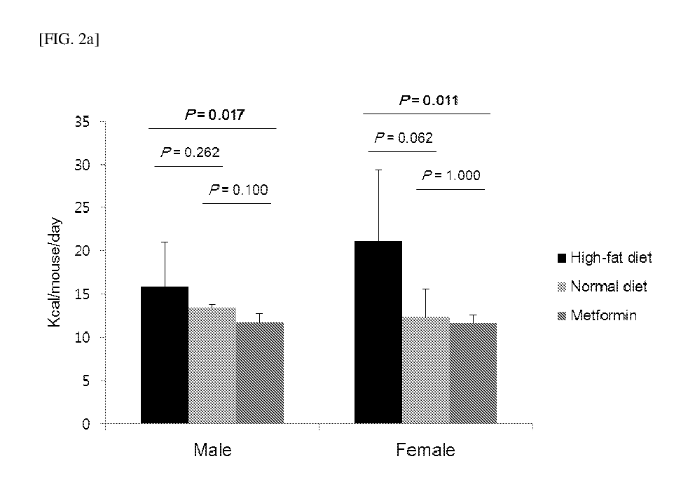

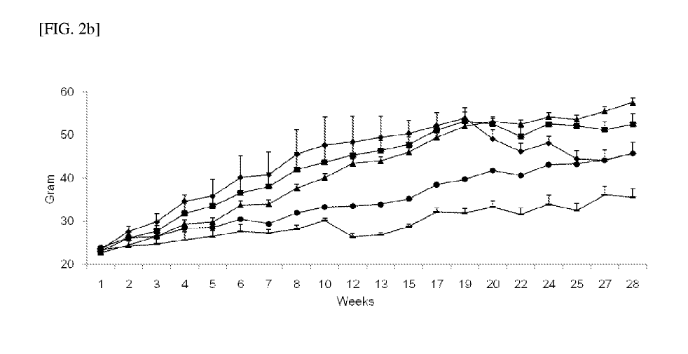

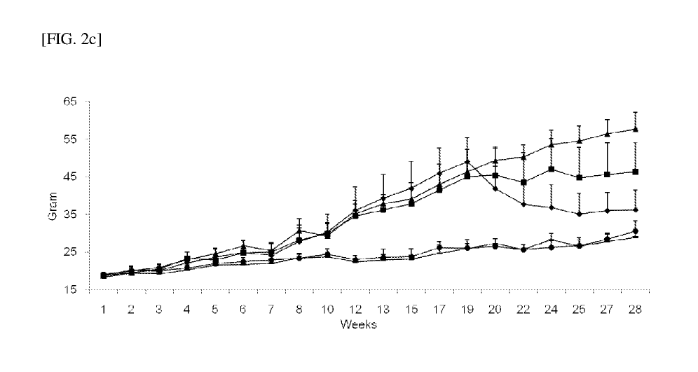

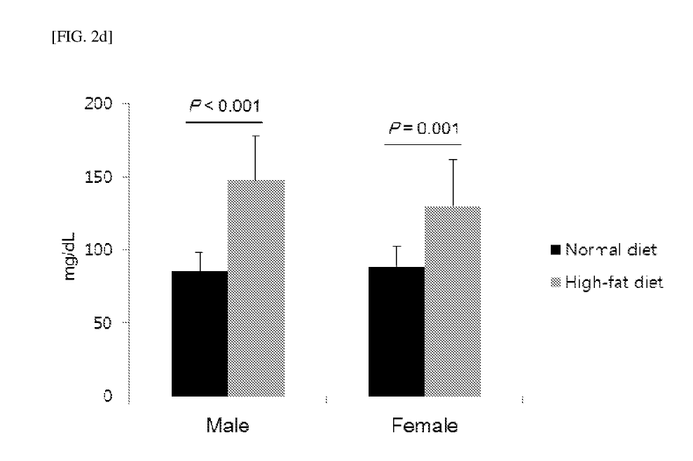

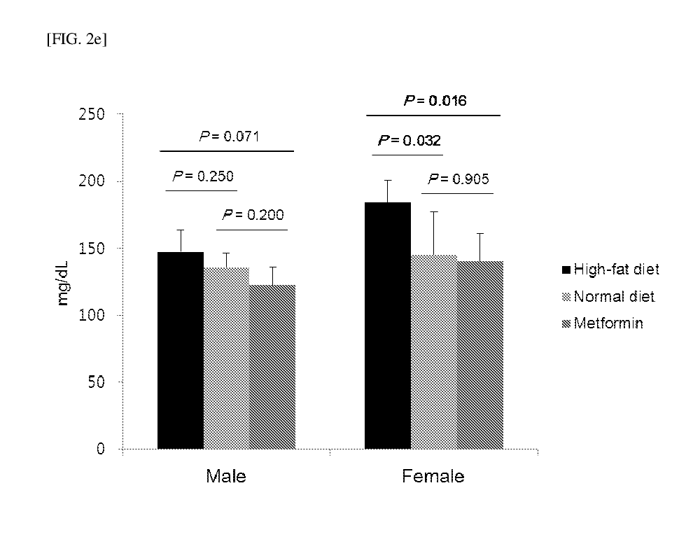

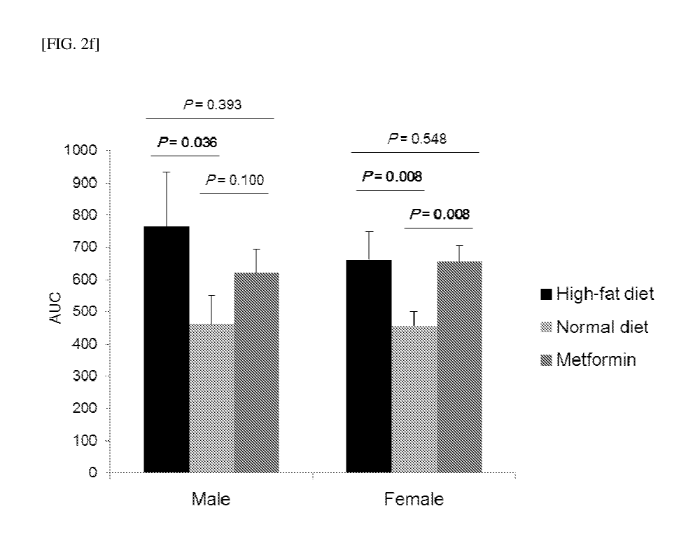

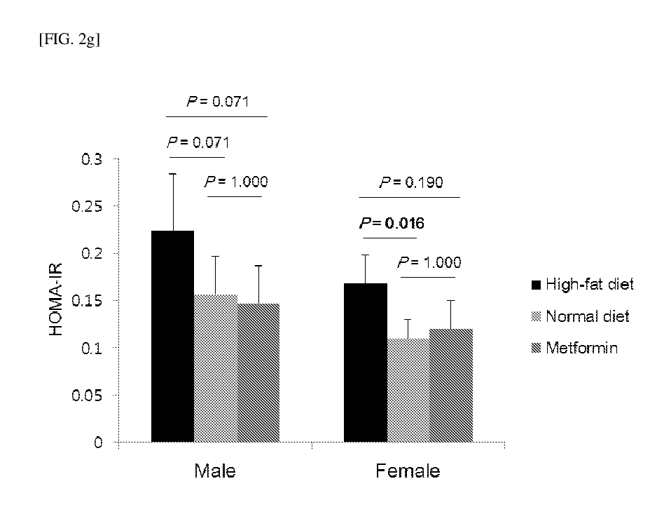

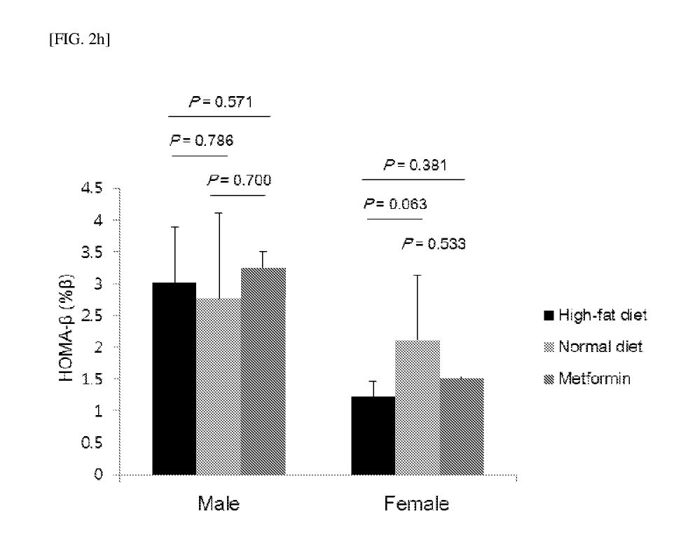

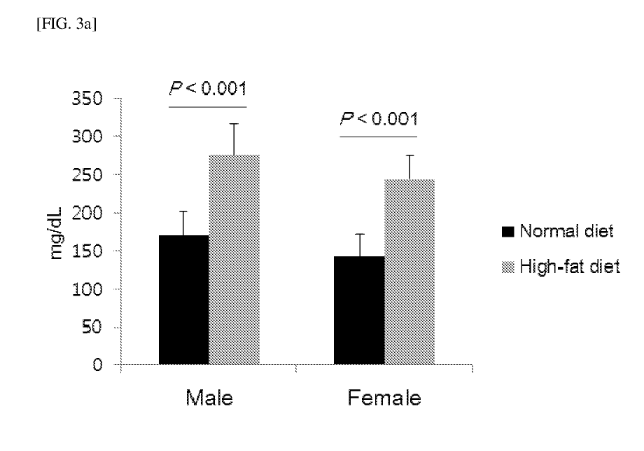

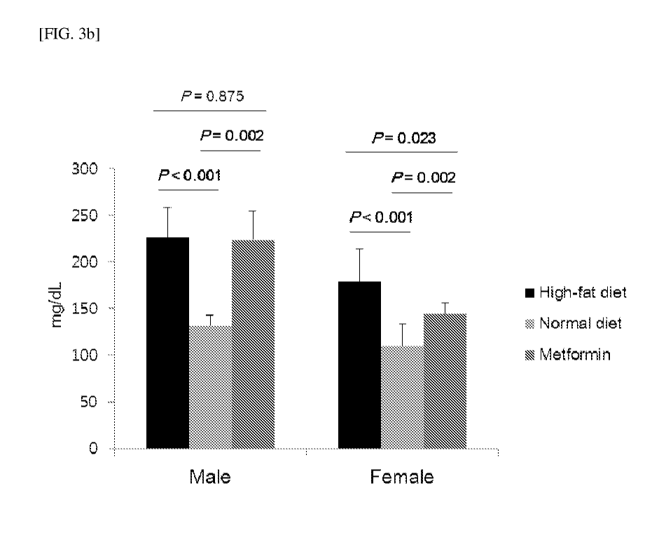

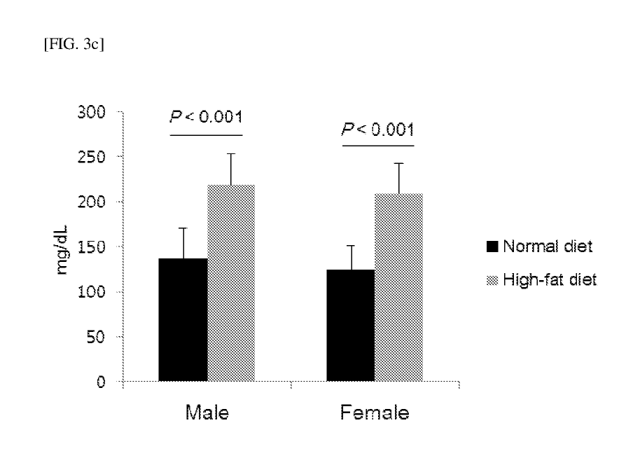

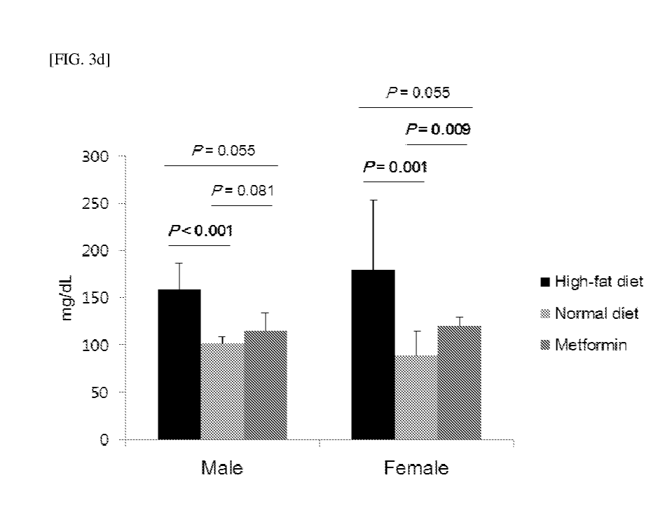

By measuring various metabolism-related indexes after high-fat diet and administration of metformin, obesity and diabetes mellitus were diagnosed and the treatment effects were evaluated. FIGS. 2a to 2h show calorie intake, body weight, and fasting glucose according to the administration of metformin and diet change. After high-fat diet, body weight and blood glucose significantly increased compared to a normal diet group, and confirmed to significantly decrease after the administration of metformin, FIGS. 3a to 3d show change in total cholesterol and high density lipoprotein, wherein total cholesterol and high density lipoprotein significantly increased due to high-fat diet, and significantly decreased by the administration of metformin and change to normal diet.

Through comparative analysis of metabolic and inflammatory factors expressed in the liver and epididymal adipose tissue, changes due to diet change and metformin administration were confirmed. FIGS. 4a to 4d relatively indicate the expression degree of metabolic factors when high-fat diet was changed to normal diet, and when metformin was administered during high-fat diet. In female, after the administration of metformin, AMPKα (P=0.023) and GLUT2 (P=0.007) decreased, and PPARα (P=0.008) increased. In male, only G6Pase significantly decreased. When high-fat diet was changed to normal diet, AMPKα (P<0.001), PPARα (P=0.029), and G6Pase (P=0.009) significantly increased. In epididymal adipose tissue, after the administration of metformin, leptin (P=0.004) and MCP-1 (P=0.008) increased in male, and only TNFα (P=0.001) significantly decreased in female. When high-fat diet was changed to normal diet, adiponectin (P=0.007), MCP-1 (P<0.001), TNFα (P<0.001), IL-6 (P<0.001) were significantly changed in female.

The expression degree of mucin genes MUC2 and MUC5 was measured in small intestine. In female, after the administration of metformin, the expression degree of both genes significantly increased.

### 2. Tissue Inspection

In the group in which high-fat diet was changed to normal diet and in the group to which metformin was administered during high-fat diet, the weights of liver and adipose tissue significantly decreased compared to the high-fat diet group. Liver steatosis also showed significant difference.

### 3. Nucleotide Sequence

In the feces samples of 40 mice, total 302,689 nucleotide sequences were secured. Among them, low quality nucleotide sequences were excluded, and total 238,522 nucleotide sequences were used for analysis. The average nucleotide sequence according to the sample was 5,963 (±1,127), and as the result of analysis, they were classified into phyla Actinobacteria, Bacteroidetes, Firmicutes, Deferribacteres, Proteobacteria, Tenericutes, and Verrucomicrobia.

### 4. Diversity in Rumen Microorganism Communities

FIG. 6 shows difference in bacterial diversity between the feces samples. First, although metformin administration during normal diet did not cause any change in diversity, microbial diversity decreased in the sample after metformin administration during high-fat diet. As the result of PCoA, change in bacterial diversity after metformin administration was distinguished from the group in which high-fat diet was changed to normal diet. And, when analogy was confirmed between bacterial communities through UniFrac distance, the difference between the group in which metformin was administered during high-fat diet and the group treated with high-fat diet only was larger than the difference between the group in which high-fat diet was changed to normal diet and the group treated with high-fat diet only.

### 5. Taxonomical Comparison of Bacteria

In the group treated with high-fat diet only, the proportion of phylum Bacteroidetes significantly decreased to 43.8±22.4% compared to the normal diet group. However, after the administration of metformin, it significantly increased to 77.5±8.7%, which is similar level to the normal diet group. To the contrary, in the group treated with high-fat diet, the proportion of phylum Firmicutes was significantly highest (50.7±19.2%). And, phylum Proteobacteria and phylum Verrucomicrobia increased at significant levels to 2.1±2.8% and 12.4±5.3% respectively, after the administration of metformin during high-fat diet. At species level, Akkermansia and Bacteroides significantly increased.

When comparing relative bacterial abundance by LDA scores, at genus level, Akkermansia, Shigella, and Blautia increased, while Clostridium decreased. When metformin was administered during normal diet, genera Clostridium and Akkermansia significantly increased identically to high-fat diet, and additionally, Alistipes increased. To the contrary, Lactobacillus iners relatively decreased.

### 6. Correlation Between Bacteria and Metabolic Factors

Using a KEGG pathway predicted from the bacteria communities in the group treated with high-fat diet only and the group to which metformin was administered during high-fat diet, correlation with various metabolic indexes was analyzed. In female, Blautia producta, Akkermansia muciniphila, and Allobaculum sp. ID4 showed significant negative correlation with body weight and fasting glucose, and in male, Blautia producta, and Akkermansia muciniphila showed negative correlation likewise, Anaerotruncus colihominis and Allobaculum sp. ID4 showed positive correlation with body weight, and Lactobacillus iners, Clostridium orbiscindens, Oscillospira guilliermondii showed positive correlation with fasting glucose. And, Blautia producta, and Akkermansia muciniphila showed negative correlation with HDL in male.

When correlation analysis was conducted by the same method as above with the group in which metformin was administered during normal diet and the group treated with normal diet only, no bacteria showed significant correlation with body weight and fasting glucose. However, Anaerotruncus colihominis, Allobaculum sp. ID4, and Lactobacillus iners showed significant negative correlation with MCP-1 and TNFα. In addition, Clostridium cocleatum showed significant positive correlation with total cholesterol, and Allobaculum sp. ID4 showed significant positive correlation with PPARα.

### 7. Correlation Between Bacteria

Akkermansia muciniphila and Blautia product showed significant positive correlation in both female and male. And, Akkermansia muciniphila showed significant positive correlation with Allobaculum sp. ID4 in female, and showed negative correlation in male. And, in male, Akkermansia muciniphila showed negative correlation with Lactobacillus iners, and Clostridium orbiscindens.

### 8. KEGG Pathway

Total 245 KEGG pathways were created using a PICRUSt method, and the created pathways were comparatively analyzed according to the groups. When metformin was administered during high-fat diet or normal diet, specific functions corresponding to the PEGG pathway significantly increased compared to the case wherein only normal diet or only high-fat diet is treated, and total 18 kinds of functions including 2 relating to amino acid metabolism (Tryptophan metabolism, and Valine, leucine and isoleucine degradation)), one relating to hydrocarbon (Ascorbate and aldarate metabolism)), 3 relating to Glycan degradation and metabolism (Glycosaminoglycan degradation, Lipopolysaccharide biosynthesis, Lipopolysaccharide biosynthesis proteins), 7 relating to fat metabolism (Biosynthesis of unsaturated fatty acids, Fatty acid elongation in mitochondria, Fatty acid metabolism, Linoleic acid metabolism, Sphingolipid metabolism, Steroid hormone biosynthesis, Synthesis and degradation of ketone bodies), one relating to Terpenoids and Polyketides metabolism (eraniol degradation), 3 relating to Xenobiotics degradation and metabolism (Bisphenol degradation, Styrene degradation, Toluene degradation), one relating to Cofactors and vitamin metabolism (Lipoic acid metabolism) significantly increased, which can be predicted as functions that increase by metformin.

1. A composition for evaluating or predicting therapeutic response of a subject to metformin, comprising an agent capable of detecting one or more microorganisms selected from the group consisting of Blautia sp, Shigella sp and Clostridium sp.

2. The composition according to claim 1, wherein the agent capable of detecting microorganisms is a microorganism-specific primer, probe, antisense oligonucleotide, aptamer or antibody.

3. The composition according to claim 2, wherein the primer is a primer capable of amplifying 16S rRNA of microorganism.

4. The composition according to claim 3, wherein the primer is a primer pair represented by SEQ ID NO: 1 and SEQ ID NO: 2, or a primer pair represented by SEQ ID NO: 3 and SEQ ID NO: 4.

5. The composition according to claim 1, wherein the patient is a patient with obesity, diabetes mellitus, or metabolic syndrome.

6. A kit for evaluating or predicting therapeutic response of a patient to metformin, comprising the composition according to claim 1.

7. A method for evaluating or predicting therapeutic response of a patient to metformin, comprising the steps of:
detecting one or more microorganisms selected from the group consisting of Blautia sp, Shigella sp and Clostridium sp from the sample of a patient before and after administration of metformin; and
determining that the patient has therapeutic response to metformin, if Blautia or Shigella increases or Clostridium decreases in the sample after administration of metformin, compared to before the administration.

8. The method according to claim 7, wherein the step of detecting microorganisms comprises the steps of:
extracting genome DNA from the sample of a patient to whom metformin is administered,
reacting the extracted genome DNA with a primer specific to one or more microorganisms selected from the group consisting of Blautia sp, Shigella sp and Clostridium sp to obtain reactant; and
amplifying the reactant.

9. The method according to claim 8, wherein the step of amplifying the reactant is conducted through polymerase chain reaction.

10. The method according to claim 7, wherein the sample of a patient is a feces sample.

11. The method according to claim 7, wherein the patient is a patient with obesity, diabetes or metabolic syndrome.

12. The method according to claim 7, wherein the microorganisms is detected by using a microorganism-specific primer, probe, antisense oligonucleotide, aptamer or antibody.

13. The method according to claim 12, wherein the primer is a primer capable of amplifying 16S rRNA of microorganism.

14. The method according to claim 13, wherein the primer is a primer pair represented by SEQ ID NO: 1 and SEQ ID NO: 2, or a primer pair represented by SEQ ID NO: 3 and SEQ ID NO: 4.

15. The method according to claim 7, wherein the method further comprises a step of administering metformin to the patient, if the patient has therapeutic response to metformin.

16. The method according to claim 7, wherein the method further comprises a step of stopping administering metformin to the patient, if the patient does not have therapeutic response to metformin.

17. A method for adjusting the dosage of metformin in a patient who has previously been administered an initial dosage of metformin, comprising:
detecting one or more microorganisms selected from the group consisting of Blautia sp, Shigella sp and Clostridium sp from the sample of a patient before and after administration of metformin; and
determining that the patient has therapeutic response to metformin, if Blautia or Shigella increases or Clostridium decreases in the sample after administration of metformin, compared to before the administration, and
administering an adjusted dosage of metformin,
wherein the adjusted dosage is greater than or equivalent to the initial dosage.
